(19) United States
(12) Patent Application Publication
(10) Pub. No.: US 2018/0104279 A1
(43) Pub. Date: Apr. 19, 2018

(54) HUMAN MILK COMPOSITIONS AND METHODS OF MAKING AND USING SAME

(71) Applicant: Prolacta Bioscience, Inc., City of Industry, CA (US)

(72) Inventors: Scott ELSTER, City of Industry, CA (US); Joseph FOURNELL, City of Industry, CA (US)

(21) Appl. No.: 15/565,874

(22) PCT Filed: Apr. 15, 2016
(86) PCT No.: PCT/US2016/027893

§ 371 Date: Oct. 11, 2017

Related U.S. Application Data
(60) Provisional application No. 62/148,024, filed on Apr. 15, 2015.

(51) Int. Cl. A61K 35/20; A61K 31/702; A61K 9/00; A61P 31/04; A61P 37/06; A23C 9/20; A23L 33/00; A23L 33/19; A23L 33/16

(52) U.S. Cl. CPC A61K 35/20; A61K 31/702; A61K 9/0053; A61K 9/0019; A61P 31/04; A23V 2002/00; A23C 9/206; A23L 33/30; A23L 33/40; A23L 33/19; A23L 33/16; A61P 37/06

(57) ABSTRACT

The disclosure features human milk compositions as well as methods of making human milk compositions and using human milk compositions. In particular, the disclosure features a method of using milk compositions to provide nutrition for subjects who are undergoing or have undergone bone marrow transplants.

### CROSS REFERENCE TO RELATED APPLICATIONS

This application claims priority to U.S. Application 62/148,024, filed Apr. 15, 2015, the entirety of which is incorporated by reference herein.

### FIELD OF THE INVENTION

The disclosure relates to human milk compositions and methods of making and using such compositions. In particular, the disclosure features methods of using human milk compositions to feed subjects who are undergoing or have undergone bone marrow transplants.

### BACKGROUND OF THE INVENTION

Medical nutrition therapy is an important consideration for patient populations at risk of malnutrition. For example, preterm infants are at risk of growth failure, developmental delays, necrotizing enterocolitis and late-onset sepsis, with the risk increasing with earlier gestational age and lower birth weight. Human milk is generally the food of choice for preterm and term infants because of its nutritional composition and immunologic benefits. The source of human milk can be, e.g., a donor or the infant's mother. Use of milk from the infant's own mother has become the preferred nutritional approach in the modern neonatal intensive care units (NICUs).

In addition, breastfeeding has been shown to protect against diarrhea with infants. Human milk contains a variety of bioactive agents including oligosaccharides that are part of the innate immune system. Oligosaccharides are the third largest solid constituent of human milk after lactose and lipid. Studies have provided evidence suggesting that human milk oligosaccharides are clinically relevant in the protection of infants with diarrhea (Morrow et al. 2004). Additional data have shown important changes in microbiota in neonatal premature infants at high-risk of necrotizing enterocolitis, mirroring the observations seen in graft versus host disease, another source of intestinal inflammation.

Another patient population at risk of malnutrition includes subjects, regardless of age, who are undergoing or have undergone bone marrow transplants (BMT). The high dose chemotherapy and/or radiotherapy performed before the transplant, along with effects of the transplant procedure itself, can lead to complications that can adversely affect the nutritional status and gut flora of these subjects. Improving the nutritional status and gut flora of these patients can lead to better outcomes.

The standard of care currently for BMT subjects who can no longer orally ingest food is total parenteral nutrition (TPN). This procedure of intravenously providing complete nutrition to a patient is convenient and facilitates administration of fluid, electrolytes and macronutrients. TPN has been shown to promote earlier engraftment and improve survival. However, these earlier studies had design flaws and with advances in the BMT procedure that significantly reduces the time until engraftment, it is questionable whether TPN is necessary in all transplant cases. TPN is associated with several potential complications including e.g. hypoglycemia, hyperglycemia, lipogenesis, hepatic complications (e.g., fatty liver, cholestasis, liver failure from steatosis), sepsis, blood clots, increased infectious complications, impaired tumor response to chemotherapy, and increased mortality. In addition, there may be adverse events associated with the central line required for TPN including the risk of central-line infection, central vein thrombosis, and damage to surrounding soft tissue and nerves.

Thus, a solution is needed to solve the problem of adequately meeting the caloric requirements of subjects who are undergoing or have undergone bone marrow transplants (BMT) that avoids the unwanted harmful side effects of TPN as well as improving the gut microbiota of the subject to provide protection.

### SUMMARY OF THE INVENTION

This disclosure features human milk compositions, e.g., pasteurized human milk compositions, and methods of making and using such compositions.

The current invention provides pasteurized human milk compositions that can be administered orally or enterally via gastric tube, oral gastric or nasojejeunal tube. The pasteurized human milk composition can be administered either alone as complete total nutrition or as supplemental nutrition to TPN. In particular, the pasteurized human milk composition can be administered to BMT subjects two years old or younger to better provide nutrition in this delicate population.

In one aspect, the disclosure features a method for providing nutrition to a subject who is undergoing or has undergone a bone marrow transplant (BMT). Another aspect of the invention is a method for improving the gut microbiota by feeding donor breast milk to young children undergoing transplant. Previous studied have identified detectable differences in microbial community composition associated with feeding breastmilk in BMT patients, and these changes may be protective against inflammation whereby the gut microbiota during bone marrow transplant could be influenced by administration of enteral donor breast milk.

In one embodiment, the method provides administering to said subject a pasteurized human milk composition comprising from about 1.5% to about 2.5% protein, from about 5% to about 6% fat, from about 7% to about 8% carbohydrates and from about 0.4% to about 3.8% human milk oligosaccharides (HMO). In another embodiment, the method provides administering to said subject a pasteurized human milk composition comprising about 2% protein, from about 5.73% to about 5.82% fat, about 7.4% carbohydrates and about 0.4% to about 3.8% HMO.

In one embodiment, the method provides administering to said subject a pasteurized human milk composition comprising from about 15 mg/mL to about 25 mg/mL protein, from about 50 mg/mL to about 60 mg/mL fat, from about 70 mg/mL to about 80 mg/mL carbohydrates and about 4 mg/mL to about 37.5 mg/mL HMO. In another embodiment, the method provides administering to said subject a pasteurized human milk composition comprising about 20.4 mg/mL protein, from about 58.48 mg/mL to about 59.39 mg/mL fat, from about 75.45 mg/mL to about 77.52 mg/mL carbohydrates and about 4 mg/mL to about 37.5 mg/mL HMO.

In one embodiment, the method provides administering to said subject a pasteurized human milk composition comprising from about 700 mg/kg/day to about 900 mg/kg/day protein, from about 2000 mg/kg/day to about 2500 mg/kg/day fat, from about 3000 mg/kg/day to about 3500 mg/kg/day carbohydrates and from about 144 mg/kg/day to about 1350 mg/kg/day of HMO. In another embodiment, the method provides administering to said subject a pasteurized human milk composition comprising about 816 mg/kg/day protein, from about 2339.2 mg/kg/day to about 2375.5 mg/kg/day fat, and from about 3019.2 mg/kg/day to about 3100.8 mg/kg/day carbohydrates.

In one embodiment, the pasteurized human milk composition provides about 90 kcal/dL. In another embodiment, the pasteurized human milk composition is provided at about 40 mL/kg/day. In another embodiment, is delivered to a subject at 32.8 kcal/kg/day and at a volume of 35 ml/kg/day.

In one embodiment, the pasteurized human milk composition further comprises immunoglobulins including secretory IgA, IgE, IgM, and/or IgG and combinations thereof. In another embodiment, the pasteurized human milk composition further comprises IgA and/or one or more constituents selected from the group consisting of: calcium, chloride, copper, iron, magnesium, manganese, phosphorus, potassium, sodium, and zinc.

In one embodiment, the pasteurized human milk composition is administered to the subject orally. In another embodiment, the pasteurized human milk composition is administered to the subject enterally via gastric tube, oral gastric or nasojejeunal tube.

In one embodiment, said subject is about two years old or younger.

In one aspect, the method comprises providing nutrition to a subject who is undergoing or has undergone BMT. In a further aspect, the method comprises administering to a subject a pasteurized human milk composition and a total parenteral nutrition (TPN) composition. In one embodiment, the human milk composition provides about 10% of the total nutrition and the TPN composition provides about 90% of the total nutrition. In another embodiment, the human milk composition provides about 40% of the total nutrition and the TPN composition provides about 60% of the total nutrition. In another embodiment, the human milk composition provides about 50% of the total nutrition and the TPN composition provides about 50% of the total nutrition. In another embodiment, the human milk composition provides about 60% of the total nutrition and the TPN composition provides about 40% of the total nutrition. In yet another embodiment, the human milk composition provides about 90% of the total nutrition and the TPN composition provides about 10% of the total nutrition. In yet another embodiment, the human milk composition provides about 100% of the total nutrition.

In one embodiment, the pasteurized human milk composition is administered orally and the TPN composition is administered intravenously. In another embodiment, the pasteurized human milk composition is administered enterally and the TPN composition is administered intravenously.

In one embodiment, the human milk composition provides about 10% of the total nutrition. In another embodiment, the human milk composition provides about 20% of the total nutrition. In still another embodiment, the human milk composition provides about 30% of the total nutrition. In still another embodiment, the human milk composition provides about 40% of the total nutrition. In still another embodiment, the human milk composition provides about 50% of the total nutrition. In still another embodiment, the human milk composition provides about 60% of the total nutrition. In still another embodiment, the human milk composition provides about 70% of the total nutrition. In still another embodiment, the human milk composition provides about 80% of the total nutrition. In these embodiments, the remainder of the nutrition not provided by the human milk composition provided herein can be from any source and will largely depend on the subject's age and severity of condition. For example, in infants who are still nursing the remainder of their nutrition may be derived from the subject's mother's own milk and/or other sources of infant nutrition including, but not limited to infant formula. In certain embodiments, the subjects condition may necessitate the use of TPN as described above. In other embodiments, the subjects are old enough and healthy enough to maintain a diet of solid food in addition to the nutrition provided by the human milk compositions featured herein.

The disclosure features standardized human milk formulations, which are produced from human milk. Methods of making and using such compositions are also described herein. Standardized human milk formulations can be supplemented with vitamins and/or minerals if desired and can be fed orally or enterally by methods described above to subjects who are undergoing or have undergone BMT. The methods of generating these compositions are designed to optimize the amount of nutrients and calories in the compositions. For example, the compositions featured herein can deliver from about 700 mg/kg/day to about 900 mg/kg/day protein, from about 2000 mg/kg/day to about 2500 mg/kg/day fat, from about 3000 mg/kg/day to about 3500 mg/kg/day carbohydrates and from about 144 mg/kg/day to about 1350 mg/kg/day of HMO. In another embodiment, the method provides administering to said subject a pasteurized human milk composition comprising about 816 mg/kg/day protein, from about 2339.2 mg/kg/day to about 2375.5 mg/kg/day fat, from about 3019.2 mg/kg/day to about 3100.8 mg/kg/day carbohydrates and from about 144 mg/kg/day to about 1350 mg/kg/day HMO.

In one aspect, the disclosure features a pasteurized human milk composition comprising: a human protein constituent from about 1.5% to about 2.5%; a human fat constituent from about 5% to about 6%; a human carbohydrate constituent from about 7% to about 8%; and a HMO constituent from about 0.4% to about 3.8%. In another aspect, the disclosure features a pasteurized human milk composition comprising: a human protein constituent of about 2%; a human fat constituent from about 5.73% to about 5.82%; a human carbohydrate constituent of about 7.4% and a HMO constituent from about 0.4% to about 3.8%. The carbohydrate constituent can include lactose. The composition can further comprise immunoglobulins including secretory IgA, IgE, IgM, and/or IgG or combinations thereof. The composition can further comprise IgA (e.g. secretory IgA) and/or one or more constituents selected from the group consisting of: calcium, chloride, copper, iron, magnesium, manganese, phosphorus, potassium, sodium, and zinc.

In one aspect, the disclosure features a pasteurized human milk composition comprising: a human protein constituent from about 15 mg/mL to about 25 mg/mL; a human fat constituent from about 50 mg/mL to about 60 mg/mL; a human carbohydrate constituent from about 70 mg/mL to about 80 mg/mL; and a HMO constituent from about 4 mg/mL to about 37.5 mg/mL. In another aspect, the disclosure features a pasteurized human milk composition comprising: a human protein constituent of about 20.4 mg/mL; a human fat constituent from about 58.48 mg/mL to about 59.39 mg/mL; a human carbohydrate constituent from about 75.45 mg/mL to about 77.52 mg/mL; and an HMO constituent of about 4 mg/mL to about 37.5 mg/mL. The carbohydrate constituent can include lactose. The composition can further comprise IgA and/or one or more constituents selected from the group consisting of: calcium, chloride, copper, iron, magnesium, manganese, phosphorus, potassium, sodium, and zinc.

In one aspect, the disclosure features a pasteurized human milk composition comprising: a human protein constituent from about 700 mg/kg/day to about 900 mg/kg/day protein, from about 2000 mg/kg/day to about 2500 mg/kg/day fat, from about 3000 mg/kg/day to about 3500 mg/kg/day carbohydrates and about 144 mg/kg/day to about 1350 mg/kg/day HMO. In another embodiment, the method provides administering to said subject a pasteurized human milk composition comprising about 816 mg/kg/day protein, from about 2339.2 mg/kg/day to about 2375.5 mg/kg/day fat, from about 3019.2 mg/kg/day to about 3100.8 mg/kg/day carbohydrates and about 144 mg/kg/day to about 1350 mg/kg/day HMO. The carbohydrate constituent can include lactose. The composition can further comprise immunoglobulins including secretory IgA, IgE, IgM, and/or IgG or combinations thereof. The composition can further comprise IgA and/or one or more constituents selected from the group consisting of: calcium, chloride, copper, iron, magnesium, manganese, phosphorus, potassium, sodium, and zinc.

The disclosure also features method of making various human milk compositions.

In one aspect, the disclosure features a method for obtaining a pasteurized human milk composition. The method includes: (a) genetically screening human milk for one or more viruses; (b) filtering the milk; (c) heat-treating the milk, e.g., at about 63° C. or greater for about 30 minutes; (d) separating the milk into cream and skim; (e) adding a portion of the cream to the skim; and (f) pasteurizing.

The genetic screening in step (a) can be polymerase chain reaction and/or can include screening for one or more viruses, e.g., human immunodeficiency virus Type 1 (HIV-1), hepatitis B virus (HBV), and/or hepatitis C virus (HCV).

The milk can be filtered through an about 200 micron screen in step (b).

The method can further include running cream, e.g., about 30-50% of cream, through a separator following step (d). In one embodiment, the method can further include filtering the skim through filters after step (d), e.g., to filter the water out of the skim. After filtering the skim after step (d), the filters used in the filtering can be washed to obtain a post wash solution. The post wash solution can be added to the skim.

The method can further include carrying out mineral analysis of the portion of the composition obtained after step (e). The method can also include adding to the composition obtained after step (e) one or more minerals selected from the group consisting of: calcium, chloride, copper, iron, magnesium, manganese, phosphorus, potassium, sodium, and zinc. Adding of the one or more minerals can include heating the composition.

The method can also include cooling the composition after step (f), carrying out biological testing of a portion of the composition after step (f), and/or carrying out nutritional testing of a portion of the composition after step (f).

The human milk of step (a) can be pooled human milk. The methods featured herein can be carried out with large volumes of the starting material, e.g., human milk, e.g., pooled human milk. The volumes can be in the range of about 75-7,500 liters/lot of starting material. In a particular embodiment, the volume is about 3,000 liters/lot. In another embodiment, the volume is about 4,000 liters/lot. In still another embodiment, the volume is about 5,000 liters/lot.

In one embodiment, the composition obtained after step (f) can include from about 1.5% to about 2.5% protein, from about 5% to about 6% fat, from about 7% to about 8% carbohydrates and from about 0.4% to about 3.8% HMO. In another embodiment, the composition obtained after step (f) can include about 2% protein, from about 5.73% to about 5.82% fat, about 7.4% carbohydrates and about 0.4% to about 3.8% HMO. In one embodiment, the composition obtained after step (f) can include protein from about 15 mg/mL to about 25 mg/mL, fat from about 50 mg/mL to about 60 mg/mL, carbohydrates from about 70 mg/mL to about 80 mg/mL and HMO from about 4 mg/mL to about 37.5 mg/mL. In a further embodiment, the composition obtained after step (f) can include protein of about 20.4 mg/mL, fat from about 58.48 mg/mL to about 59.39 mg/mL, carbohydrate from about 75.45 mg/mL to about 77.52 mg/mL and HMO from about 4 mg/mL to about 37.5 mg/mL. In one embodiment, the composition obtained after step (f) can include protein from about 700 mg/kg/day to about 900 mg/kg/day protein, from about 2000 mg/kg/day to about 2500 mg/kg/day fat, from about 3000 mg/kg/day to about 3500 mg/kg/day carbohydrates and from about 144 mg/kg/day to about 1350 mg/kg/day. In another embodiment, the method provides administering to said subject a pasteurized human milk composition comprising about 816 mg/kg/day protein, from about 2339.2 mg/kg/day to about 2375.5 mg/kg/day fat, from about 3019.2 mg/kg/day to about 3100.8 mg/kg/day carbohydrates and about 144 mg/kg/day to about 1350 mg/kg/day HMO.

In another aspect, the disclosure features a method for obtaining a pasteurized human milk composition. The method includes: (a) genetically screening human milk for one or more viruses; (b) filtering the milk; (c) adding cream; and (d) pasteurizing.

In one embodiment, the genetic screening in step (a) can be polymerase chain reaction. The genetic screening can include screening for one or more viruses, e.g., HIV-1, HBV, and/or HCV.

The milk can be filtered through an about 200 micron screen in step (b). The method can further include ultra-filtering the whole milk after step (b) through filters. The filters used during ultra-filtering can be post washed.

The composition can be cooled after step (d). Biological and/or nutritional testing of the composition can be carried out after step (d).

Human milk of step (a) can be pooled human milk. The methods featured herein can be carried out with large volumes of the starting material, e.g., human milk, e.g., pooled human milk. The volumes can be in the range of about 75-7,500 liters/lot of starting material. In a particular embodiment, the volume is about 3,000 liters/lot. In another embodiment, the volume is about 4,000 liters/lot. In still another embodiment, the volume is about 5,000 liters/lot.

The method can also include adding to the composition obtained after step (c) one or more minerals selected from the group consisting of: calcium, chloride, copper, iron, magnesium, manganese, phosphorus, potassium, sodium, and zinc.

In one embodiment, the composition obtained after step (d) can include from about 1.5% to about 2.5% protein, from about 5% to about 6% fat, from about 7% to about 8% carbohydrates and from about 0.4% to about 3.8% HMO. In another embodiment, the composition obtained after step (d) can include about 2% protein, from about 5.73% to about 5.82% fat, about 7.4% carbohydrates, from about 0.4% to about 3.8% HMO. In one embodiment, the composition obtained after step (d) can include protein from about 15 mg/mL to about 25 mg/mL, fat from about 50 mg/mL to about 60 mg/mL, carbohydrates from about 70 mg/mL to about 80 mg/mL and HMO from about 4 mg/mL to about 37.5 mg/mL. In a further embodiment, the composition obtained after step (d) can include protein of about 20.4 mg/mL, fat from about 58.48 mg/mL to about 59.39 mg/mL, carbohydrate from about 75.45 mg/mL to about 77.52 mg/mL and HMO from about 4 mg/mL to about 37.5 mg/mL. In one embodiment, the composition obtained after step (d) can include protein from about 700 mg/kg/day to about 900 mg/kg/day protein, from about 2000 mg/kg/day to about 2500 mg/kg/day fat, from about 3000 mg/kg/day to about 3500 mg/kg/day carbohydrates from 144 mg/kg/day to about 1350 mg/kg/day. In another embodiment, the method provides administering to said subject a pasteurized human milk composition comprising about 816 mg/kg/day protein, from about 2339.2 mg/kg/day to about 2375.5 mg/kg/day fat, from about 3019.2 mg/kg/day to about 3100.8 mg/kg/day carbohydrates and from about 144 mg/kg/day to about 1350 mg/kg/day HMO.

In certain embodiments, a method is provided for optimizing gut flora in a subject undergoing a BMT by administering a pasteurized human milk composition. In particular embodiments, the optimization of gut flora includes increasing diversity of gut flora. In certain embodiments, optimizing gut flora includes increasing the level of lactobacillus species. In certain embodiments, a method is provided for decreasing pathogenic bacteria in the gut by administering a pasteurized human milk composition. In another embodiment, a method is provided for decreasing the incidence and/or severity of GVHD in a subject receiving a bone marrow transplant by providing the subject a pasteurized human milk composition. In certain embodiments, the human milk composition comprises: 1.5% to about 2.5% protein, from about 5% to about 6% fat, from about 7% to about 8% carbohydrates and from about 0.4% to about 3.8% HMO or protein from about 15 mg/mL to about 25 mg/mL, fat from about 50 mg/mL to about 60 mg/mL, carbohydrates from about 70 mg/mL to about 80 mg/mL and HMO from about 4 mg/mL to about 37.5 mg/mL or protein from about 700 mg/kg/day to about 900 mg/kg/day protein, from about 2000 mg/kg/day to about 2500 mg/kg/day fat, from about 3000 mg/kg/day to about 3500 mg/kg/day carbohydrates from 144 mg/kg/day to about 1350 mg/kg/day. In certain embodiments, the composition is provided at about 30 kcal/kg/day to about 40 kcal/kg/day.

### BRIEF DESCRIPTION OF THE DRAWINGS

FIG. 1 is a graph depicting reduction in the levels of soluble IL2r during milk administration.

FIG. 2 is a flow diagram showing an overview of the study design.

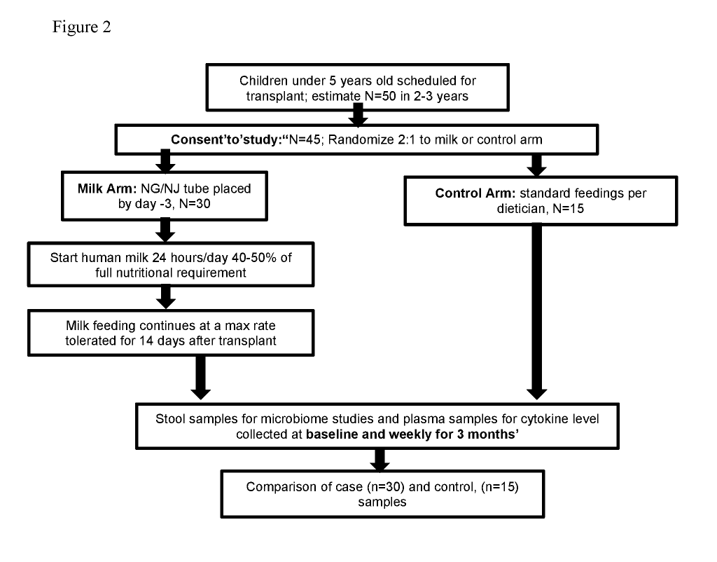

### DETAILED DESCRIPTION OF THE INVENTION

This disclosure features human milk compositions, e.g., pasteurized human milk compositions, and methods of making and using such compositions.

The disclosure also features standardized human milk formulations, which are produced from human milk. Methods of making and using such compositions are also described. These standardized human milk formulations can be used to feed, e.g., subjects who are undergoing or have undergone bone marrow transplants, without mixing them with other fortifiers or milk. These standardized human milk formulations can also be used to provide said subjects with a human-derived nutritional formulation that can substitute for or supplement total parenteral nutrition (TPN). Human milk formulations can contain various caloric contents, for example, the human milk compositions described herein can provide about 30-40 kcal/kg/day.

The compositions of the present disclosure are generated from human donor milk, e.g., pooled milk, which undergoes rigorous genetic screening, processing (e.g., to concentrate nutrients in the fortifier compositions, and/or to reduce bioburden), and pasteurization. The milk can be supplemented with various minerals and/or vitamins. Thus, the disclosure also features methods of obtaining and processing milk from human donors.

Total parenteral nutrition (TPN), a process of providing nutrition intravenously and bypassing the gastrointestinal tract, is often used to feed subjects who have undergone BMT. However, TPN is associated with several potential complications including e.g. hypoglycemia, hyperglycemia, lipogenesis, hepatic complications (e.g., fatty liver, cholestasis, liver failure from steatosis), sepsis, blood clots, increased infectious complications, impaired tumor response to chemotherapy, and increased mortality. In addition, there may be adverse events associated with the central line required for TPN including the risk of central-line infection, central vein thrombosis, and damage to surrounding soft tissue and nerves. Enteral feeding, or providing nutrition, directly to the stomach, duodenum or jejunum is associated with fewer infections, is considered more physiologic and less expensive. Accordingly, it is desirable to provide said subject with enteral nutrition as soon as possible rather than TPN, in order to avoid the negative effects associated with TPN. The human milk compositions described herein can provide the needed caloric content for said subjects. Maintaining a fully human milk based diet reduces the incidence of complications such as necrotizing enterocolitis in infants, for example. Therefore, it is contemplated that oral or enteral feeds of pasteurized human milk compositions may be used in place of TPN or to supplement TPN, as enteral feeding is often combined with TPN.

The methods of the present disclosure can be used to process large volumes of donor milk, e.g., about 75-7,500 liters/lot of starting material. In a particular embodiment, the volume is about 3,000 liters/lot. In another embodiment, the volume is about 4,000 liters/lot. In still another embodiment, the volume is about 5,000 liters/lot.

As used herein, the term “adulterant” refers to any non-human milk found in human milk. The addition of adulterants to human milk is referred to as “adulteration”. Examples of adulterants include milk from non-human species (e.g., cow milk, goat milk, etc.), milk-like products from plants (e.g., soy milk) and infant formula.

As used herein, the term “bone marrow transplant” or “BMT” refers to a therapeutic procedure that involves chemotherapy and/or radiotherapy followed by intravenous infusion of hematopoietic stem cells to reestablish marrow function in subjects with damaged or defective bone marrow. Other terms that may be used to refer to the same procedure include “stem cell transplant” and “hematopoietic stem cell transplant.” The procedure is used to treat a variety of oncologic, hematologic, immunologic and hereditary diseases. There are two major types of BMT: allogeneic, where the marrow or blood cells are received from a donor other than the patient, and autologous, where the patient's own marrow or blood cells are used. A rare type of allogeneic transplant, syngeneic, refers to the donation of genetically identical hematopoietic stem cells from one identical twin to the other.

As used herein, the term “human oligosaccharide” or “milk oligosaccharide” or “human milk oligosaccharide” or “HMO” refers to unconjugated complex carbohydrates that are highly abundant in human milk. HMOs are diverse soluble oligosaccharides, carbohydrate polymers formed from a small number of monosaccharides.

As used herein, the term “contaminant” refers to the inclusion of unwanted substances in human milk. While an adulterant is a “contaminant” generally the use of the term “contaminant” as used herein generally refers to other substances such as drugs, environmental pollutants and/or bacteria and viruses. The inclusion of contaminants to human milk is referred to as “contamination.” The inclusion of contaminants may be due to any reason including but not limited to accident, negligence or intent.

As used herein, the terms “donor” and “individual” are used interchangeably and refer to a woman who supplies or provides a volume of her breast milk, regardless of whether or not she is compensated, e.g., monetarily, for the milk.

As used herein, the term “enteral feeding” refers to the delivery of a nutritionally complete feed, containing protein, carbohydrate, fat, water, minerals and vitamins, directly into the stomach, duodenum or jejunum. Short-term access is usually done with nasogastric (NG) or nasojejeunal (NJ) tubes. Percutaneous endoscopic gastrostomy (PEG) or jejunostomy placement can be used for feedings longer than one month.

As used herein, the terms “human milk”, “breast milk”, “donor milk”, and “mammary fluid” are used interchangeably and refer to milk from a human.

As used herein, the term “parenteral nutrition” refers to feeding a person for part or all of the nutritional needs intravenously, bypassing the usual process of eating and digestion. The nutritional formulae contain nutrients such as glucose, amino acids, lipids, vitamins and dietary minerals. Total parenteral nutrition (TPN) occurs when no significant nutrition is obtained by other routes. Peripheral parenteral nutrition is administered through vein access in a limb rather than through a central vein.

As used herein, the term “whole milk” refers to human milk from which no fat has been removed.

As used herein, the term “bioburden” refers to microbiological contaminants and pathogens (generally living) that can be present in milk, e.g., viruses, bacteria, mold, fungus and the like.

All patents, patent applications, and references cited herein are incorporated in their entireties by reference. Unless defined otherwise, technical and scientific terms used herein have the same meaning as that commonly understood by one of skill in the art.

### Bone Marrow Transplants

A bone marrow transplant (BMT) is a therapeutic procedure that involves chemotherapy and/or radiotherapy followed by intravenous infusion of hematopoietic stem cells to reestablish marrow function in subjects with damaged or defective bone marrow. Diseased or damaged stem cells can arise from a number of disorders including: genetic conditions, hematologic malignancies (e.g. leukemias, myelomas, lymphomas); solid tumors (breast cancer, glioma, and non-small-cell lung cancer); other pathologic conditions (β-thalassemia, autoimmune disorders, and hereditary metabolic disorders).

Bone marrow transplants can be allogeneic or autologous. In allogeneic BMT, the marrow or blood cells are received from a donor other than the patient. Donor blood cells should closely match the genetic background of the recipient to minimize graft rejection of the host, or graft versus host disease. In autologous BMT, the patient's own marrow or blood cells are used. Hematopoietic stem cells can be collected from peripheral blood, bone marrow or collected cord blood.

BMT is preceded by a conditioning regimen that involves high doses of chemotherapy and/or radiotherapy. This conditioning may serve several purposes, including elimination of the cancer, making space in the bone marrow for new cells to grow and suppression of the immune system so that new cells may be accepted. Therefore, a patient who is “undergoing BMT” as used herein is meant a subject who is being prepared for bone marrow transplant, for example, a patient who is undergoing a conditioning regimen involving chemotherapy and/or radiotherapy.

### Nutritional Requirements of Subjects Undergoing Bone Marrow Transplants

The nutritional status of a subject who is undergoing or has undergone BMT is severely affected by both the conditioning regimen of chemotherapy and/or radiotherapy before the transplant and by the transplant procedure itself. In addition, for subjects such as children, there is also the requirement to maintain growth and development. It has been estimated that the energy requirements of BMT patients reach 130%-150% of predicted basal energy expenditure.

Conditioning regimens involving high doses of chemotherapy or radiotherapy are associated with gastrointestinal (GI) toxicities such as colitis, neutropenic colitis, gastritis, duodenitis, oroesophageal mucositis, nausea, vomiting and diarrhea. In mucositis, the integrity of the mucosal epithelia lining the oral cavity, esophagus and GI tract are denuded, which can lead to increased infection, malabsorption, diarrhea and pain. Thus these regimens can render challenging the maintenance of adequate nutrition.

Subjects who have undergone an allogeneic transplant are susceptible to graft versus host disease (GVHD). The phenomenon of GVHD occurs due to the presence of immunologically competent donor cells in an immuno-incompetent host. In other words, the host is unable to destroy the donor cells due to lack of immune function, but the donor cells attack the host as they see the host as foreign. GVHD can be acute or chronic, depending upon the timing of onset of symptoms. Changes to skin, GI and other organs develop that lead to complications such as persistent nausea, anorexia, diarrhea, oral sensitivity and steatorrhea (excess fat in feces indicative of fat malabsorption). Thus the transplant procedure itself causes complications that negatively affect maintenance of adequate nutrition.

The present disclosure features human milk compositions and methods of making and using such compositions for feeding subjects who are undergoing or have undergone BMT. The particular human milk compositions herein provide a unique balance of protein, fat, carbohydrates and HMO such that useful calories can be delivered without the need for large volumes of liquid. The compositions described herein, by virtue of their HMO content, have the additional benefit of optimizing gut flora and protecting against GVHD in subjects undergoing bone marrow transplant. The human milk compositions can be used instead of or to supplement total parenteral nutrition (TPN). The compositions can be supplemented with various vitamins and/or minerals. The composition can further comprise immunoglobulins including secretory IgA, IgE, IgM, and/or IgG and combinations thereof. The compositions can also contain IgA (e.g., secretory IgA) and various components described herein.

### Human Milk Compositions

The compositions featured herein contain various amounts of nutrients, e.g., protein, carbohydrates, fat, vitamins, and minerals, as well as other milk components, such as immunoglobulins, lactoferrin, oligosaccharides, and lysozyme. Standardized human milk formulations can be supplemented with vitamins and/or minerals if desired and can be fed orally or enterally to subjects who are undergoing or have undergone BMT. The methods of generating these compositions are designed to optimize the amount of nutrients and calories in the compositions. For example, the compositions featured herein can deliver from about 700 mg/kg/day to about 900 mg/kg/day protein, from about 2000 mg/kg/day to about 2500 mg/kg/day fat, from about 3000 mg/kg/day to about 3500 mg/kg/day carbohydrates and from about 144 mg/kg/day to about 1350 mg/kg/day HMO. In another embodiment, the compositions featured herein can deliver from about 816 mg/kg/day protein, from about 2339.2 mg/kg/day to about 2375.5 mg/kg/day fat, from about 3019.2 mg/kg/day to about 3100.8 mg/kg/day carbohydrates and from about 144 mg/kg/day to about 1350 mg/kg/day HMO.

### Standardized Human Milk Formulations

The standardized human milk formulations featured herein can be used in lieu of or to supplement TPN for subjects who are undergoing or have undergone BMT. They include various nutritional components for subject growth and development.

In one embodiment, the standardized human milk formulation can include: a human protein constituent from about 1.5% to about 2.5%; a human fat constituent from about 5% to about 6%; a human carbohydrate constituent from about 7% to about 8%; and a HMO constituent from about 0.4% to about 3.8%. In another embodiment, the standardized human milk formulation can include: a human protein constituent of about 2%; a human fat constituent from about 5.73% to about 5.82%; a human carbohydrate constituent of about 7.4%; and an HMO constituent from about 0.4% to about 3.8%. The carbohydrate constituent can include lactose.

In one embodiment, the standardized human milk formulation can include: a human protein constituent from about 15 mg/mL to about 25 mg/mL; a human fat constituent from about 50 mg/mL to about 60 mg/mL; a human carbohydrate constituent from about 70 mg/mL to about 80 mg/mL and a HMO constituent from about 4 mg/mL to about 37.5 mg/mL. In another embodiment, the standardized human milk formulation can include: a human protein constituent of about 20.4 mg/mL; a human fat constituent from about 58.48 mg/mL to about 59.39 mg/mL; a human carbohydrate constituent from about 75.45 mg/mL to about 77.52 mg/mL; and a HMO constituent from about 4 mg/mL to about 37.5 mg/mL. The carbohydrate constituent can include lactose. In one embodiment, the total caloric content of the formulations can be, e.g., from about 0.100 kcal/mL to about 1.500 kcal/mL. In another embodiment, the total caloric content of the formulations can be from about 0.100 kcal/mL to about 1.250 kcal/mL. In another embodiment, the total caloric content of the formulations can be from about 0.100 kcal/mL to about 1.000 kcal/mL. In a further embodiment, the total caloric content of the formulations can be about 0.900 kcal/mL. In one embodiment, the total caloric content of the formulations can be about 91 kcal/dL.

In one embodiment, the standardized human milk formulation can include: a human protein constituent from about 700 mg/kg/day to about 900 mg/kg/day protein, from about 2000 mg/kg/day to about 2500 mg/kg/day fat, from about 3000 mg/kg/day to about 3500 mg/kg/day carbohydrates and from about 144 mg/kg/day to about 1350 mg/kg/day HMO. In another embodiment, the standardized human milk formulation can include: a human protein constituent of from about 816 mg/kg/day protein, from about 2339.2 mg/kg/day to about 2375.5 mg/kg/day fat, from about 3019.2 mg/kg/day to about 3100.8 mg/kg/day carbohydrates; and from about 144 mg/kg/day to about 1350 mg/kg/day HMO. The carbohydrate constituent can include lactose.

The milk formulation can be supplemented with vitamins and/or minerals. In one embodiment, the composition can include: calcium concentration of about 0.40-1.50 mg/mL; chloride concentration of about 0.30-0.80 mg/mL; copper concentration of about 0.0005-0.0021 mg/mL; iron concentration of about 0.001-0.005 mg/mL; magnesium concentration of about 0.03-0.13 mg/mL; manganese concentration of about 0.01-0.092 mg/mL; phosphorus concentration of about 0.15-0.631 mg/mL (e.g., about 0.15-0.60 mg/mL); potassium concentration of about 0.60-1.20 mg/mL; sodium concentration of about 0.20-0.60 mg/mL; and zinc concentration of about 0.0025-0.0120 mg/mL.

### Specific Components of the Featured Compositions

One component of the milk compositions featured herein is protein. In the body, protein is needed for growth, synthesis of enzymes and hormones, and replacement of protein lost from the skin, urine and feces. These metabolic processes determine the need for both the total amount of protein in a feeding and the relative amounts of specific amino acids. The adequacy of the amount and type of protein in a feeding for subjects is determined by measuring growth, nitrogen absorption and retention, plasma amino acids, certain blood analytes and metabolic responses. Some proteins present in the featured compositions beneficial for other than purely nutritional reasons include human IgA, lysozyme, and lactoferrin.

Another constituent of the milk compositions described herein is fat. Fat is generally a source of energy for subjects, not only because of its high caloric density but also because of its low osmotic activity in solution.

HMOs are another important constituent of the human milk compositions featured herein. While HMOs have diverse actions, HMOs play an important role in increasing the diversity and otherwise optimizing gut flora. The optimization of gut flora in turn leads to a decrease in pathogenic bacterial infections of the gut as well as an overall decrease in gut inflammation which is a contributor to the pathogenesis of GVHD. Thus, the HMOs delivered as a part of the human milk compositions described herein decrease the incidence and/or severity of GVHD. In certain embodiments, feeding subjects undergoing BMT with the human milk compositions described herein prevents the onset of GVHD. In certain embodiments, feeding subjects undergoing BMT with the human milk compositions described herein decrease the severity of GVHD.

Vitamins and minerals are important to proper nutrition and development of subjects. A subject requires electrolytes, e.g., sodium, potassium and chloride for growth and for acid-base balance. Sufficient intakes of these electrolytes are also needed for replacement of losses in the urine and stool and from the skin. Calcium, phosphorus and magnesium are needed for proper bone mineralization and growth.

Trace minerals are associated with cell division, immune function and growth. Consequently, sufficient amounts of trace minerals are needed for subject growth and development. Some trace minerals that are important include, e.g., copper, magnesium and iron (which is important, e.g., for the synthesis of hemoglobin, myoglobin and iron-containing enzymes). Zinc is needed, e.g., for growth, for the activity of numerous enzymes, and for DNA, RNA and protein synthesis. Copper is necessary for, e.g., the activity of several important enzymes. Manganese is needed, e.g., for the development of bone and cartilage and is important in the synthesis of polysaccharides and glyoproteins. Accordingly, the human milk formulations and compositions of the invention can be supplemented with vitamins and minerals as described herein.

Vitamin A is a fat-soluble vitamin essential for, e.g., growth, cell differentiation, vision and proper functioning of the immune system. Vitamin D is important, e.g., for absorption of calcium and to a lesser extent, phosphorus, and for the development of bone. Vitamin E (tocopherol) prevents peroxidation of polyunsaturated fatty acids in the cell, thus preventing tissue damage. Folic acid plays a role in, e.g., amino acid and nucleotide metabolism.

As described above, the variability of human milk vitamin and mineral concentrations often require some fortification to insure that a child is receiving adequate amounts of vitamins and minerals. Examples of vitamins and minerals that can be added to the human milk compositions featured herein include: vitamin A, vitamin B1, vitamin B2, vitamin B6, vitamin B12, vitamin C, vitamin D, vitamin E, vitamin K, biotin, folic acid, pantothenic acid, niacin, m-inositol, calcium, phosphorus, magnesium, zinc, manganese, copper, sodium, potassium, chloride, iron and selenium. The compositions can also be supplemented with: chromium, molybdenum, iodine, taurine, carnitine and choline may also require supplementation.

The osmolality of standardized human milk formulations featured herein can affect adsorption, absorption, and digestion of the compositions. High osmolality, e.g., above about 400 mOsm/Kg H₂O, has been associated with increased rates of necrotizing enterocolitis (NEC), a gastrointestinal disease that affects neonates (see, e.g., Srinivasan et al., Arch. Dis. Child Fetal Neonatal Ed. 89:514-17, 2004). The osmolality of the human milk compositions of the disclosure is typically less than about 400 mOsm/Kg H₂O. Typically the osmolality is from about 310 mOsm/Kg of water to about 380 mOsm/Kg of water. The osmolality can be adjusted by methods known in the art.

### Methods of Making Human Milk Compositions

The human milk compositions described herein are produced from whole human milk. The human milk may be obtained from an infant's own mother or from one or more donors. In certain embodiments, the human milk is pooled to provide a pool of human milk. For example, a pool of human milk comprises milk from two or more (e.g., ten or more) donors. As another example, a pool of human milk comprises two or more donations from one donor.

Obtaining Human Milk from Qualified and Selected Donors

Generally, human milk is provided by donors, and the donors are pre-screened and approved before any milk is processed. Various techniques are used to identify and qualify suitable donors. A potential donor must obtain a release from her physician and her child's pediatrician as part of the approval process. This helps to insure, inter alia, that the donor is not chronically ill and that her child will not suffer as a result of the donation(s). Methods and systems for qualifying and monitoring milk collection and distribution are described, e.g., in U.S. patent application Ser. No. 12/728,811 (U.S. 2010/0268658), which is incorporated herein by reference in its entirety. Donors may or may not be compensated for their donation.

Usually, donor screening includes a comprehensive lifestyle and medical history questionnaire that includes an evaluation of prescription and non-prescription medications, testing for drugs of abuse, and serological testing for certain pathogens. The donor is screened for, e.g., human immunodeficiency virus Type 1 (HIV-1), HIV-2, human T-lymphotropic virus Type 1 (HTLV-I), HTLV-II, hepatitis B virus (HBV), hepatitis C virus (HCV), and syphilis.

Donors may be periodically requalified. For example, a donor is required to undergo screening by the protocol used in their initial qualification every four months, if the donor wishes to continue to donate. A donor who does not requalify or fails qualification is deferred until such time as they do, or permanently deferred if warranted by the results of requalification screening. In the event of the latter situation, all remaining milk provided by that donor is removed from inventory and destroyed or used for research purposes only.

A donor may donate at a designated facility (e.g., a milk bank office) or, in a preferred embodiment, express milk at home. If the donor will be expressing milk at home, she will measure the temperature in her freezer with, e.g., a supplied thermometer to confirm that it is cold enough to store human milk in order to be approved.

### Testing Donor Identity

Once the donor has been approved, donor identity matching may be performed on donated human milk because the milk may be expressed by a donor at her home and not collected at a milk banking facility. In a particular embodiment, each donor's milk can be sampled for genetic markers, e.g., DNA markers, to guarantee that the milk is truly from the approved donor. Such subject identification techniques are known in the art (see, e.g., International Application Serial No. PCT/US2006/36827 which is incorporated herein by reference in its entirety). The milk may be stored (e.g., at −20° C. or colder) and quarantined until the test results are received.

For example, the methods featured herein may include a step for obtaining a biological reference sample from a potential human breast milk donor. Such sample may be obtained by methods known in the art such as, but not limited to, a cheek swab sample of cells, or a drawn blood sample, milk, saliva, hair roots, or other convenient tissue. Samples of reference donor nucleic acids (e.g., genomic DNA) can be isolated from any convenient biological sample including, but not limited to, milk, saliva, buccal cells, hair roots, blood, and any other suitable cell or tissue sample with intact interphase nuclei or metaphase cells. The sample is labeled with a unique reference number. The sample can be analyzed at or around the time of obtaining the sample for one or more markers that can identify the potential donor. Results of the analysis can be stored, e.g., on a computer-readable medium. Alternatively, or in addition, the sample can be stored and analyzed for identifying markers at a later time.

It is contemplated that the biological reference sample may be DNA typed by methods known in the art such as short tandem repeat (STR) analysis of STR loci found throughout the genome, HLA analysis of HLA loci or multiple gene analysis of individual genes/alleles. The DNA-type profile of the reference sample is recorded and stored, e.g., on a computer-readable medium.

It is further contemplated that the biological reference sample may be tested for self-antigens using antibodies known in the art or other methods to determine a self-antigen profile. The antigen (or another peptide) profile can be recorded and stored, e.g., on a computer-readable medium.

A test sample of human milk is taken for identification of one or more identity markers. The sample of the donated human milk is analyzed for the same marker or markers as the donor's reference sample. The marker profiles of the reference biological sample and of the donated milk are compared. The match between the markers (and lack of any additional unmatched markers) would indicate that the donated milk comes from the same individual as the one who donated the reference sample. Lack of a match (or presence of additional unmatched markers) would indicate that the donated milk either comes from a non-tested donor or has been contaminated with fluid from a non-tested donor.

The donated human milk sample and the donated reference biological sample can be tested for more than one marker. For example, each sample can be tested for multiple DNA markers and/or peptide markers. Both samples, however, need to be tested for at least some of the same markers in order to compare the markers from each sample.

Thus, the reference sample and the donated human milk sample may be tested for the presence of differing identity marker profiles. If there are no identity marker profiles other than the identity marker profile from the expected subject, it generally indicates that there was no fluid (e.g., milk) from other humans or animals contaminating the donated human milk. If there are signals other than the expected signal for that subject, the results are indicative of contamination. Such contamination will result in the milk failing the testing.

The testing of the reference sample and of the donated human milk can be carried out at the donation facility and/or milk processing facility. The results of the reference sample tests can be stored and compared against any future donations by the same donor.

### Screening for Contaminants and Adulterants

The milk is then tested for pathogens. The milk may be genetically screened, e.g., by polymerase chain reaction (PCR), to identify, e.g., viruses, such as HIV-1, HBV and HCV. Additionally, a microorganism panel that screens via culture for various bacterial species, fungus and mold may be used to detect contaminants. For example, a microorganism panel may test for aerobic count, Bacillus cereus, Escherichia coli, Salmonella, Pseudomonas, coliforms, Staphylococcus aureus, yeast and mold. Pathogen screening may be performed both before and after pasteurization.

In addition to screening for pathogens, the donor milk may also be tested for drugs of abuse (e.g., cocaine, opiates, synthetic opioids (e.g. oxycodone/oxymorphone) methamphetamines, benzodiazepine, amphetamines, and THC).

The donor milk may also be screened for one or more adulterants. Adulterants include any non-human milk fluid or filler that is added to a human milk donation, thereby causing the donation to no longer be unadulterated, pure human milk. Particular adulterants to be screened for include non-human milk and infant formula. As used herein, “non-human milk” refers to both animal-, plant- and synthetically-derived milks. Examples of non-human animal milk include, but are not limited to, buffalo milk, camel milk, cow milk, donkey milk, goat milk, horse milk, reindeer milk, sheep milk, and yak milk. Examples of non-human plant-derived milk include, but are not limited to, almond milk, coconut milk, hemp milk, oat milk, rice milk, and soy milk. Examples of infant formula include, cow milk formula, soy formula, hydrolysate formula (e.g., partially hydrolyzed formula or extensively hydrolyzed formula), and amino acid or elemental formula. Cow milk formula may also be referred to as dairy-based formula. In particular embodiments, the adulterants that are screened for include cow milk, cow milk formula, goat milk, soy milk, and soy formula.

Methods known in the art may be adapted to detect non-human milk proteins, e.g., cow milk and soy proteins, in a human milk sample. In particular, immunoassays that utilize antibodies specific for a protein found in an adulterant that is not found in human milk can be used to detect the presence of the protein in a human milk sample. For example, an enzyme-linked immunosorbent assay (ELISA), such as a sandwich ELISA, may be used to detect the presence of an adulterant in a human milk sample. An ELISA may be performed manually or be automated. Another common protein detection assay is a western blot, or immunoblot. Flow cytometry is another immunoassay technique that may be used to detect an adulterant in a human milk sample. ELISA, western blot, and flow cytometry protocols are well known in the art and related kits are commercially available. Another useful method to detect adulterants in human milk is infrared spectroscopy and in particular mid-range Fourier transform infrared spectrometry (FTIR).

The human milk may be pooled prior to screening. In one embodiment, the human milk is pooled from more than one donation from the same individual. In another embodiment, the human milk is pooled from two or more, three or more, four or more, five or more, six or more, seven or more, eight or more, nine or more, or ten or more individuals. In a particular embodiment, the human milk is pooled from ten or more individuals. The human milk may be pooled prior to obtaining a sample by mixing human milk from two or more individuals. Alternatively, human milk samples may be pooled after they have been obtained, thereby keeping the remainder of each donation separate.

The screening step will yield a positive result if the adulterant is present in the human milk sample at about 20% or more, about 15% or more, about 10% or more, about 5% or more, about 4% or more, about 3% or more, about 2% or more, about 1% or more, or about 0.5% or more of the total volume of the milk donation.

The screening of the donated human milk for one or more adulterants can be carried out at the donation facility and/or milk processing facility.

Human milk that has been determined to be free of an adulterant, or was found to be negative for the adulterant, is selected and may be stored and/or further processed. Human milk that contains an adulterant will be discarded and the donor may be disqualified. For example, if an adulterant is found in two or more human milk samples from the same donor, the donor is disqualified.

### Processing Human Milk

Once the human milk has been screened, it is processed to produce a high fat product, e.g., a human cream composition. The donation facility and milk processing facility can be the same or different facility. Processing of milk can be carried out with large volumes of human milk, e.g., about 75 liters/lot to about 7,500 liters/lot of starting material. In a particular embodiment, the volume is about 3,000 liters/lot. In another embodiment, the volume is about 4,000 liters/lot. In still another embodiment, the volume is about 5,000 liters/lot.

Methods of obtaining compositions that include lipids from human milk to provide nutrition to patients are described in PCT Application PCT/US07/86973 filed on Dec. 10, 2007 (WO 2008/073888), the contents of which are incorporated herein in their entirety.

After the human milk is carefully analyzed for both identification purposes and to avoid contamination and/or adulteration as described above, the milk then undergoes filtering, e.g., through about a 200 micron filter, and heat treatment. For example, the composition can be treated at about 63° C. or greater for about 30 minutes or more. Next, the milk is transferred to a separator, e.g., a centrifuge, to separate the cream (i.e., the fat portion) from the skim. The skim can be transferred into a second processing tank where it remains at about 2 to 8° C. until a filtration step. Optionally, the cream separated from the skim, can undergo separation again to remove more skim.

Following the separation of cream and skim, the skim portion undergoes further filtration, e.g., ultrafiltration. This process concentrates the nutrients in the skim milk by filtering out the water. The water obtained during the concentration is referred to as the permeate. The resulting skim portion can be further processed to produce human milk fortifiers and/or standardized human milk formulations.

### Use of Human Milk Compositions

The disclosed pasteurized human milk compositions are particularly useful for providing nutrition for subjects who are undergoing or have undergone BMT in order to provide enough calories to meet the increased nutritional requirements associated with insult to the gastrointestinal tract as a result of the conditioning regimen before BMT, the complications resulting from the BMT procedure and the demands of physical growth of subjects such as children. Further, due to their HMO content, the pasteurized human milk fortifiers of the present invention are also useful in optimizing human gut flora, decreasing the incidence of bacterial infections of the gut and decreasing the incidence and/or severity of GVHD in patients undergoing BMT. TPN is often used to feed subjects who have undergone BMT. The use of human lipids for parenteral nutrition, a practice of intravenous feeding (e.g., total parenteral nutrition), for a patient in need thereof is described in PCT Application PCT/US07/86973 filed on Dec. 10, 2007 (WO 2008/073888), the contents of which are incorporated herein in their entirety. However, due to the negative effects associated with TPN, enteral feeding may be desired. Enteral feeding can also be combined with TPN.

The pasteurized human milk compositions described herein may be used as complete or supplemental nutrition. Accordingly, the pasteurized human milk compositions described herein may be administered orally (e.g., bottle feeding) or enterally (e.g. nasogastric tube feeding) with or without supplementation with total parenteral nutrition (TPN). Therefore, in one embodiment, a pasteurized human milk composition and a total parenteral nutrition (TPN) composition is administered to a subject who is undergoing or has undergone BMT. One of skill in the art will understand that the percentage value of the human milk composition can be any non-zero percentage of the total nutrition up to 100%. The percentage of the TPN composition will be a value that when added to the human milk composition percentage totals 100%. For example, in one embodiment, the human milk composition provides about 40% of the total nutrition and the TPN composition provides about 60% of the total nutrition. In another embodiment, the human milk composition provides about 100% of the total nutrition. In yet another embodiment, the human milk composition provides about 50% of the total nutrition and the TPN composition provides about 50% of the total nutrition. In another embodiment, the pasteurized human milk composition is administered orally and the TPN composition is administered intravenously. In another embodiment, the pasteurized human milk composition is administered enterally and the TPN composition is administered intravenously.

In one embodiment, the pasteurized human milk composition administered to a subject who is undergoing or has undergone BMT comprises from about 1.5% to about 2.5% protein, from about 5% to about 6% fat, from about 7% to about 8% carbohydrates and from about 0.4% to about 3.8% HMO. In another embodiment, the pasteurized human milk composition administered to a subject who is undergoing or has undergone BMT comprises about 2% protein, from about 5.73% to about 5.82% fat, about 7.4% carbohydrates and from about 0.4% to about 3.8% HMO.

In one embodiment, the pasteurized human milk composition administered to a subject who is undergoing or has undergone BMT comprises from about 15 mg/mL to about 25 mg/mL protein, from about 50 mg/mL to about 60 mg/mL fat, from about 70 mg/mL to about 80 mg/mL carbohydrates, and from about 4 mg/mL to about 37.5 mg/mL HMO. In another embodiment, the pasteurized human milk composition administered to a subject who is undergoing or has undergone BMT comprises about 20.4 mg/mL protein, from about 58.48 mg/mL to about 59.39 mg/mL fat, from about 75.45 mg/mL to about 77.52 mg/mL carbohydrates and from about 4 mg/mL to about 37.5 mg/mL HMO.

In one embodiment, the pasteurized human milk composition administered to a subject who is undergoing or has undergone BMT comprises from about 700 mg/kg/day to about 900 mg/kg/day protein, from about 2000 mg/kg/day to about 2500 mg/kg/day fat, from about 3000 mg/kg/day to about 3500 mg/kg/day carbohydrates and from about 144 mg/kg/day to about 1350 mg/kg/day HMO. In another embodiment, the pasteurized human milk composition administered to a subject who is undergoing or has undergone BMT comprises about 816 mg/kg/day protein, from about 2339.2 mg/kg/day to about 2375.5 mg/kg/day fat, from about 3019.2 mg/kg/day to about 3100.8 mg/kg/day carbohydrates and from about 144 mg/kg/day to about 1350 mg/kg/day HMO.

In one embodiment, the pasteurized human milk composition administered to a subject who is undergoing or has undergone BMT can further comprise immunoglobulins including secretory IgA, IgE, IgM, and/or IgG and combinations thereof. In one embodiment, the pasteurized human milk composition administered to a subject who is undergoing or has undergone BMT can further comprise IgA and/or one or more constituents selected from the group consisting of: calcium, chloride, copper, iron, magnesium, manganese, phosphorus, potassium, sodium, and zinc.

In one embodiment, the pasteurized human milk composition is administered at about 30 to about 40 kcal/kg/day to a subject who is undergoing or has undergone BMT. In another embodiment, the pasteurized human milk composition is administered at about 30 to about 40 mL/kg/day to said subject. In another embodiment, the milk has a target caloric content of 91 kcal/dl and is delivered to subjects at 32.8 kcal/kg/day and at a volume of 35 ml/kg/day.

In one embodiment, the pasteurized human milk composition is administered to a subject who is five years old or younger and is undergoing or has undergone BMT. In another embodiment, the subject is two years old or younger and undergoing or has undergone BMT.

In certain embodiments, the pasteurized human milk compositions of the present invention, by virtue of their HMO content decrease the incidence and/or severity of and/or prevent pathogenic bacterial infections of the gut associated with a decreased diversity of gut flora. In other embodiments, the pasteurized human milk compositions of the present invention, by virtue of their HMO content, increase the diversity of gut flora. In certain embodiments, the frequency and/or predominance of lactobacillales is increased in subjects who are administered the pasteurized human milk compositions of the current invention. In other embodiments, the pasteurized human milk compositions of the present invention, by virtue of their HMO content, decrease the incidence and/or severity of and/or prevent GVHD.

All documents cited herein are expressly incorporated by reference in their entireties for all purposes.

### EXAMPLES

### Example 1

A pilot feasibility study showed that administration of enteral human milk to children undergoing BMT is feasible, with none of the children requiring discontinuation of milk, and led to change in the gut microbiome compared with those receiving conventional feeding. Moreover, children receiving milk show reduction in a key plasma inflammatory marker compared with conventionally fed children. Ten children received enteral human milk continuously from 3 days before to 14 days after bone marrow transplantation. Stool samples were collected from subjects using a standardized protocol. Samples were classified as having been collected pre-treatment (baseline), or approximately day 10 and day 20 post-treatment.

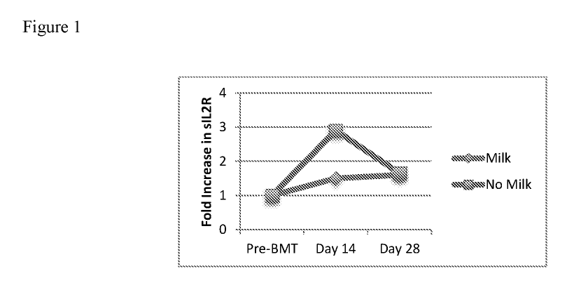

After completion of the pilot study, a microbial community analysis of banked samples was undertaken. A standardized extraction protocol was used to extract DNA from stool sample. Sequencing of the v4 region of 16s rRNA gene was performed at Broad Institute using Illumina MiSeq with universal primers. The human milk fed group was compared to controls at each time point. Samples did not differ in microbial community composition at baseline. However, at day 10 and 20 post-treatment time points, the microbial communities of intervention and control children differed significantly based on analysis blinded to study group. At day 10, the intervention group (group B) was found to have less family Streptococcaceae (genus Streptococcus) and less family Actinomycetaceae (genus Actinomyces) than controls (group A). At day 20, the intervention group had fewer Streptococcus anginosus, fewer organisms of the order Clostridiales (family Clostridiaceae, genus Clostridium, species C. perfringens), and fewer organisms of the phylum Bacteroidetes than controls. Also at day 20, 3 of the 10 intervention group children had detectable levels of Acinetobacter rhizospherae, whereas none of the 4 control group children had this organism. Thus, detectable differences in microbial community composition associated with this study were identified, and it is anticipated that these changes may be protective against inflammation. Moreover, levels of soluble IL2r, a commonly used marker of inflammation and GVHD were reduced during milk administration compared with conventionally fed children (FIG. 1).

Taken together, these observations demonstrate that the gut microbiota during bone marrow transplant is influenced by administration of enteral donor breast milk.

Study Procedures

The eligibility for enrollment in the study are 1) children less than 5 years old receiving transplant (autologous or allogeneic) and 2) Parents must give informed consent.

The expected duration of the study is 2-3 years.

The primary study endpoint is the composition of the gut microbiome 21 days after transplant. The intervention will be considered promising for further study if there is greater diversity and more frequent predominance of lactobacillales in the intervention group compared with the conventionally fed children.

Secondary study endpoints include production of pro-inflammatory cytokines, frequency of bacteremia, and frequency of diarrhea causing bowel infections (e.g. c. diff, norovirus).

Donor Milk

The donor milk used is made from human milk that is pooled from 300 mothers and then pasteurized prior to use. Milk donors are screened using the conventional criteria used for screening blood donors. In addition, all donors must be taking no drugs or medication at the time of milk donation. The human milk is processed such that it contains specific protein, fat, carbohydrate and HMO content. It is designed for use in BMT patients up to 5 years of age, for whom it will provide 40 to 50% of their macronutrient requirements, and is expected to provide 40 to 50% of full nutrient requirements for most infants 6 to 12 months of age.

Enteral milk feeding will commence on day −3 and can be given orally or by NG or NJ tube. While milk could be drunk orally from a bottle we expect that the large majority of children will need placement of a feeding tube as this is usual for any source of enteral nutrition during transplant. If enteral feeds are not tolerated, nutrition will be provided intravenously per standard practice.

Feeding will be supervised and advanced as quickly as tolerated with a goal of providing 40-50% of nutritional needs from the donor milk.

Donor milk feeding will continue through day 14 after transplant, and will then be discontinued once a satisfactory sample for microbiome studies has been obtained.

The goal will be to maintain enteral feeding between day −3 and day +14, but it is recognized that the volume of enteral feeds will need to be adjusted per patient tolerance. Diarrhea is a usual event post-BMT and the standard BMT diagnostic order set will be used to identify any specific enteric pathogens per standard practice.

Randomization

Participants will be randomized to either the milk or control arm (2:1, milk:control). It is anticipated that 30 participants will be randomized to the milk arm and 15 participants to the control arm.

Children randomized to the control arm will receive standard enteral or parenteral nutrition per standard clinical practice, supervised by the same registered dietician.

The study coordinator will hold 45 envelopes (30 envelopes for the milk arm and 15 envelopes for the control arm). The coordinator will provide one envelope to the registered dietician when a participant is enrolled.

Children of breastfeeding mothers will not be randomized and their enrollment will not be part of the randomized cohort.

Study Observations

Sample collection will continue weekly until day +100 and then monthly as possible for the first year. Although enteral feeding with milk will end at day 14, we wish to observe how long any changes in the microbiome persist.

Sample Collection:

Baseline blood (plasma, serum, and peripheral blood mononuclear cells), urine and stool of patients undergoing transplant

Repeat blood (plasma, serum, and peripheral blood mononuclear cells), urine and stool samples weekly through day +100

Repeat blood (plasma, serum, and peripheral blood mononuclear cells), urine and stool samples monthly as possible for the first year

Repeat blood (plasma, serum, and peripheral blood mononuclear cells), urine and stool samples of all patients with any event (ICU admission, relapse, etc.).

The BMT division has 2.5 full time employees dedicated to sample collection, processing and storage. The laboratory is located in the R building. All children transplanted at CCHMC are offered enrollment on a BMT repository protocol, and more than 90% consent. Weekly stool urine and blood samples are collected on these children for the first 3 months after transplant, according to the same schedule proposed in this study. The same infrastructure used for the repository will be used for this study.

Blood collection may be spaced over 2 days of the week as needed to ensure that the amount of blood collected is not excessive. All children will have in-dwelling venous access, and no venipunctures will be performed for sample collection.

Subject Enrollment

The parents of all children under the age of 5 years receiving transplant will be invited to participate in the donor milk study. It is anticipated that about 1 in 4 will agree to participate. Children whose parents decline consent for the donor milk study will be fed according to standard practice, under the guidance of the BMT unit dieticians.

Statistical Analysis

The primary study endpoint is the diversity of the microbiome at day 21 post-transplant. Bar charts will be prepared representing the distribution of bacterial classes in stool samples. It is expected that the percent of lactobacillales will be higher in children receiving donor milk than those without. Bacterial diversity will be quantified using the Shannon index and bacterial chaos using the Bray-Curtis time index (Jenq et al, 2012, Magurran, 2004). It is expected that recipients of enteral donor milk will have greater diversity and less chaos than those conventionally fed. Production of pro-inflammatory cytokines will be compared between cases and controls. We expect that there will be higher levels of pro-inflammatory cytokines in conventionally fed children compared with recipients of donor human milk, in particular sIL2r. Median and Range fold increase above baseline for each cytokine will be calculated for cases and for controls at weekly time-points. These values will be tested for statistical significance using the Wilcoxon Rank Sum test. In the pilot data the fold increase of siLR2 levels in the control group were approximately 2 times the fold increase in the cases. The log fold increase of siLR2 levels between Day 14 and baseline from the pilot study were used to determine that a sample of 30 cases and 15 controls will have a 0.87 power to detect if the fold increase in siL2r levels is greater in controls than in cases with a 0.05 level of significance. Additional data regarding occurrence of GVHD and bacterial sepsis will be collected prospectively and stored in the BMT database per routine practice. The frequencies of GVHD and bacterial sepsis will be compared between cases and controls. We expect that frequencies of GVHD and bacterial infection will be lower in recipients of donor human milk than in conventionally fed infants. Fisher's Exact test will be used to examine the difference in frequency of the categorical variables between cases and controls.

Production of pro-inflammatory cytokines will be compared between cases and controls. We expect that there will be higher levels of pro-inflammatory cytokines in conventionally fed children compared with recipients of donor human milk. Plasma biomarkers will be examined for the cytokines, including those in Table 1. Mean fold increase above baseline for each cytokine will be calculated for cases and for controls at weekly time-points, and results compared. Additional biomarkers may be tested.

TABLE 1

| Pro-inflammatory | Anti-inflammatory |
|---|---|
| IL-1b | IL-10 |
| IL-6 | |
| IL-8 | |
| Il-18 | |
| IFN-γ | |
| MIF | |
| MIP-1b | |
| MCP-1 | |
| TNFR1 | |
| sIL2R | |
| TNF-α | |

What is claimed is:

1. A method for providing nutrition to a subject who is undergoing or has undergone a bone marrow transplant (BMT), the method comprising administering to said subject a pasteurized human milk composition comprising from about 1.5% to about 2.5% protein, from about 5% to about 6% fat, from about 7% to about 8% carbohydrates and from about 0.4 to about 3.8% human milk oligosaccharides (HMO).

2. The method of claim 1, wherein the pasteurized human milk composition comprises about 2% protein, from about 5.73% to about 5.82% fat, about 7.4% carbohydrates and about 0.4% to about 3.8% HMO.

3. The method of claim 1, wherein the pasteurized human milk composition can further comprise one or more secretory immunoglobulins selected from the group consisting of IgA, IgE, IgM, and IgG and/or one or more constituents selected from the group consisting of: calcium, chloride, copper, iron, magnesium, manganese, phosphorus, potassium, sodium, and zinc.

4. The method of claim 1, wherein the pasteurized human milk composition is administered at about 30 kcal/kg/day to about 40 kcal/kg/day.

5. The method of claim 4 wherein the pasteurized human milk composition is administered at about 32 kcal/kg/day to about 33 kcal/kg/day.

6. The method of claim 1, wherein the pasteurized human milk composition is administered at about 30 mL/kg/day to about 40 mL/kg/day.

7. The method of claim 1, wherein the pasteurized human milk composition is administered orally or enterally.

8. The method of claim 1, wherein the subject is about five years old or younger.

9. The method of claim 8, wherein the subject is about two years old or younger.

10. The method of claim 1, wherein the pasteurized human milk composition comprises pooled donor milk.

11. A method of providing nutrition to a subject who is undergoing or has undergone BMT, the method comprising administering to said subject a pasteurized human milk composition and a total parenteral nutrition (TPN) composition, wherein the pasteurized human milk composition comprises from about 1.5% to about 2.5% protein, from about 5% to about 6% fat, from about 7% to about 8% carbohydrates and from about 0.4% to about 3.8% HMO.

12. The method of claim 11, wherein the pasteurized human milk composition comprises about 2% protein, from about 5.73% to about 5.82% fat, about 7.4% carbohydrates and from about 0.4% to about 3.8% HMO.

13. The method of claim 11 or 12 wherein the human milk composition provides about 10% of the total nutrition and the TPN composition provides about 90% of the total nutrition.

14. The method of claim 11 or 12, wherein the human milk composition provides about 40% of the total nutrition and the TPN composition provides about 60% of the total nutrition.

15. The method of claim 11 or 12, wherein the human milk composition provides about 50% of the total nutrition and the TPN composition provides about 50% of the total nutrition.

16. The method of claim 11 or 12, wherein the human milk composition provides about 60% of the total nutrition and the TPN composition provides about 40% of the total nutrition.

17. The method of claim 11 or 12, wherein the human milk composition provides about 90% of the total nutrition and the TPN composition provides about 10% of the total nutrition.

18. The method of claim 11 or 12, wherein the pasteurized human milk composition is administered at about 30 kcal/kg/day to about 40 kcal/kg/day.

19. The method of claim 11 or 12 wherein the pasteurized human milk composition is administered at about 32 kcal/kg/day to about 33 kcal/kg/day.

20. The method of claim 11 or 12, wherein the pasteurized human milk composition is administered at about 30 mL/kg/day to about 40 mL/kg/day.

21. The method of claim 11, wherein the pasteurized human milk composition is administered orally or enterally and the TPN composition is administered intravenously.

22. The method of claim 11 wherein the subject is about five years old or younger.

23. The method of claim 11, wherein the subject is about two years old or younger.

24. The method of claim 11, wherein the pasteurized human milk composition comprises pooled donor milk.

25. A pasteurized human milk composition comprising: a human protein constituent from about 1.5% to about 2.5%; a human fat constituent from about 5% to about 6%; a human carbohydrate constituent from about 7% to about 8% and an HMO constituent from about 0.4% to about 3.8%.

26. The pasteurized human milk composition of claim 25, wherein the human protein constituent is about 2%; the human fat constituent is from about 5.73% to about 5.82%; the human carbohydrate constituent is about 7.4% and the HMO constituent is about 0.4% to about 3.8%.

27. The pasteurized human milk composition of claim 25 or 26, wherein the carbohydrate constituent further includes lactose.

28. The pasteurized human milk composition of claim 25 or 26, wherein the composition further comprises IgA and/or one or more constituents selected from the group consisting of: calcium, chloride, copper, iron, magnesium, manganese, phosphorus, potassium, sodium, and zinc.

29. A method for increasing the diversity of gut flora in subjects who have undergone or who are undergoing BMT comprising enterally administering a pasteurized human milk composition comprising a human protein constituent from about 1.5% to about 2.5%; a human fat constituent from about 5% to about 6%; a human carbohydrate constituent from about 7% to about 8% and an HMO constituent from about 0.4% to about 3.8%.

30. A method for preventing pathogenic bacterial infections of the gut in subjects who have undergone or who are undergoing BMT comprising enterally administering a pasteurized human milk composition comprising a human protein constituent from about 1.5% to about 2.5%; a human fat constituent from about 5% to about 6%; a human carbohydrate constituent from about 7% to about 8% and an HMO constituent from about 0.4% to about 3.8%.

31. A method for preventing graft versus host disease (GVHD) in subjects who have undergone or who are undergoing BMT comprising enterally administering a pasteurized human milk composition comprising a human protein constituent from about 1.5% to about 2.5%; a human fat constituent from about 5% to about 6%; a human carbohydrate constituent from about 7% to about 8% and an HMO constituent from about 0.4% to about 3.8%.

32. The method of any one of claims 29-31, wherein the pasteurized human milk composition comprises about 2% protein, from about 5.73% to about 5.82% fat, about 7.4% carbohydrates and about 0.4% to about 3.8% HMO.

33. The method of any one of claims 29-31, wherein the pasteurized human milk composition can further comprise one or more secretory immunoglobin selected from the group consisting of IgA, IgE, IgM, and IgG and/or one or more constituents selected from the group consisting of: calcium, chloride, copper, iron, magnesium, manganese, phosphorus, potassium, sodium, and zinc.

34. The method of any one of claims 29-31, wherein the pasteurized human milk composition is administered at about 30 kcal/kg/day to about 40 kcal/kg/day.

35. The method of claim 34 wherein the pasteurized human milk composition is administered at about 32 kcal/kg/day to about 33 kcal/kg/day.

36. The method of any one of claims 29-31, wherein the pasteurized human milk composition is administered at about 30 mL/kg/day to about 40 mL/kg/day.

37. The method of any one of claims 29-31, wherein the subject is about five years old or younger.

38. The method of claim 37, wherein the subject is about two years old or younger.

39. The method of any one of claims 29-31, wherein the pasteurized human milk composition comprises pooled donor milk.
